(19) United States
(12) Patent Application Publication
(10) Pub. No.: US 2018/0153935 A1
(43) Pub. Date: Jun. 7, 2018

(54) Medicinal Composition for Total Body Detoxification

(71) Applicant: Lisa Companioni Smith, Tampa, FL (US)

(72) Inventor: Lisa Companioni Smith, Tampa, FL (US)

(21) Appl. No.: 15/832,224

(22) Filed: Dec. 5, 2017

Related U.S. Application Data
(60) Provisional application No. 62/430,242, filed on Dec. 5, 2016.

(51) Int. Cl. A61K 33/44; A61K 31/714; A61K 33/14; A61K 36/9066

(52) U.S. Cl. CPC A61K 33/44; A61K 36/9066; A61K 33/14; A61K 31/714

(57) ABSTRACT

A medicinal composition for total body detoxification is administered to a user to remove toxins from the user's body, as well as, provide hangover relief subsequent to the consumption of alcoholic beverages. The medicinal composition includes a quantity of activated charcoal and a quantity of dietary supplements; the quantity of activated charcoal and the quantity of dietary supplements being heterogeneously mixed into an orally-administrable composition. The orally-administrable composition is consumed twice a day or proportionally to the quantity of alcohol consumed. The quantity of activated charcoal binds to toxins during digestion to prevent the uptake of the toxins into the user's body. The quantity of dietary supplements replenishes beneficial nutrients to the user.

The current application claims a priority to the U.S. Provisional Patent application Ser. No. 62/430,242 filed on Dec. 5, 2016.

### FIELD OF THE INVENTION

The present invention relates generally to medicinal compositions. More, specifically, the present invention relates to a medicinal composition to be imbibed orally to detoxify the user's body.

### BACKGROUND OF THE INVENTION

Toxins may enter a person's body in a plurality of ways, one of which is orally. Foods are known to contain a plurality of toxins from sources such as pesticides, processing, or that occur naturally. While toxins are generally not present in harmful levels in food products, excessively consuming foods containing toxins is detrimental to a person's wellbeing. Removing toxins from the body before the toxins are metabolized helps to prevent complications related to high concentrations of the toxins.

Alcohol consumption has always been quite prevalent throughout human history, and today is no exception. However, excessive alcohol consumption often causes uncomfortable hangovers from dehydration that can be difficult to cope with. One of the common tactics of dealing with the dehydration is the consumption of water to replenish the body. The issue with this tactic is that the alcohol consumer is often feeling the ailments from dehydration before the water is consumed. Alternatively, the alcohol consumer could preempt the hangover by consuming more water or less alcohol when drinking. This requires a level of foresight that the alcohol consumer may not anticipate from the strength of the beverages consumed or from impaired judgement due to the consumption of alcohol.

Therefore, it is an object of the present invention to remove toxins from the user's body, as well as, provide relief for hangovers from the excessive consumption of alcohol. The present invention is a medicinal composition for total body detoxification that is imbibed by the user to remove toxins and replenish nutrients to the user. The present invention incorporates activated charcoal to bind to toxins and prevent the toxins from being metabolized.

### DETAIL DESCRIPTIONS OF THE INVENTION

The present invention is a medicinal composition for total body detoxification. The present invention filters and absorbs toxins present within the digestive tract. The present invention is intended to be administered orally to a patient twice a day to cleanse the patient's body from toxins; however, the present invention has applications for hangover relief when the present invention is administered to the patient subsequent to the patient's consumption of alcohol.

The present invention comprises a quantity of activated charcoal and a quantity of dietary supplements. The quantity of activated charcoal is ingested to absorb toxins, trapping the toxins within the quantity of activated charcoal. Therefore, the quantity of activated charcoal prevents the absorption of the toxins with the patient's body by binding to the toxins before the toxins are metabolized. The quantity of activated charcoal is preferred to be derived from coconuts, as this type of activated charcoal contains ultrafine grains to bind with toxins more effectively. The quantity of dietary supplements imparts beneficial nutrients or biological effects to the user. The quantity of activated charcoal and the quantity of dietary supplements are heterogeneously mixed into an orally-administrable composition. The orally administrable composition is administered twice by mouth after a meal or consuming an alcoholic beverage to remove or prevent toxins from being absorbed in digestion. The orally-administrable composition is preferred to be between 280 milligrams (mg) and 320 mg.

In accordance to Table 1, the quantity of activated charcoal is between 82 percent by weight (wt %) and 95 wt % of the orally-administrable composition. The activatable charcoal is incorporated as a significant portion of the orally-administrable composition in order to sufficiently bind to any harmful drugs or toxins present in the user's body.

TABLE 1
Orally-administrable Composition

| Component | Approximate wt. % |
|---|---|
| Activated Charcoal | 82-95 |
| Dietary Supplements | 5-18 |

In some embodiments of the present invention, the quantity of dietary supplements is preferred to be cyanocobalamin, more commonly known as vitamin B12. Cyanocobalamin is often used to treat pernicious anemia, vitamin B12 deficiency, thyrotoxicosis, hemorrhage, malignancy, liver disease and kidney disease. Additionally, in some embodiments, the present invention comprises a quantity of turmeric root powder. The quantity of turmeric root powder imparts anti-inflammatory and antioxidant properties to the patient. The quantity of turmeric root powder is heterogeneously mixed into the orally-administrable composition. In accordance to Table 2, the quantity of cyanocobalamin is preferred to be approximately 0.32 wt % of the orally administrable mixture. The quantity of turmeric root powder is preferred to be approximately 16 wt % of the orally administrable mixture. This composition provides a sufficient quantity of cyanocobalamin and a sufficient quantity of turmeric root powder to impart the respective beneficial properties for detoxifying the user's body.

TABLE 2
Orally-administrable Composition

| Component | Approximate wt. % |
|---|---|
| Activated Charcoal | 82-95 |
| Dietary Supplements | |
| Cyanocobalamin | 0.32 |
| Turmeric Root Powder | 16 |

In accordance to alternate embodiments for the quantity of dietary supplements, the quantity of dietary supplements is selected from the group consisting of a quantity of sodium chloride, a quantity of potassium chloride, a quantity of magnesium oxide, and combinations thereof. The quantity of sodium chloride and the quantity of potassium chloride provide electrolytes to the user's body that may be lost from dehydration. The quantity of magnesium oxide provides heartburn and indigestion relief. Detailed in Table 3, a specific alternate embodiment of the dietary supplements is a combination of the quantity of sodium chloride, the quantity of potassium chloride, and the quantity of magnesium oxide. The quantity of sodium chloride is approximately 1.7 wt % of the orally-administrable composition. The quantity of potassium chloride is approximately 3.5 wt % of the orally-administrable composition. The quantity of magnesium chloride is approximately 3.2 wt % of the orally-administrable composition. This composition provides a sufficient quantity of sodium chloride, quantity of potassium chloride, and quantity of magnesium oxide to increase the user's electrolyte levels, prevent indigestion and prevent heartburn without being harmful to the user.

TABLE 3
Orally-administrable Composition

| Component | Approximate wt. % |
|---|---|
| Activated Charcoal | 82-95 |
| Dietary Supplements | |
| Sodium Chloride | 1.7 |
| Potassium Chloride | 3.5 |
| Magnesium Oxide | 3.2 |

In the alternate embodiments of quantity of dietary supplement, the present invention is preferred to be administered immediately after consuming alcohol or before the user goes to sleep after consuming alcohol to reduce the likelihood that the user experiences a hangover. The orally-administrable composition is administered proportionally to the quantity of alcohol consumed by the user. If the user has consumed from two to four alcoholic beverages, the user should ingest four of the present invention to experience hangover relief. If the user has consumed from four to six alcoholic beverages, the user should ingest four to six of the present invention to experience hangover relief. The user should not exceed ingesting more than twelve of the present invention daily.

The present invention is preferred to be manufactured without the use of yeast, corn, wheat, soy, gluten, milk, sugar, preservatives, artificial colors, artificial flavors, or artificial sweeteners. Manufacturing the present invention without the aforementioned compounds reduces the possibility for the user to experience an allergic reaction to the present invention. Additionally, the lack of incorporation for the aforementioned compounds in the present invention prevents the user from consuming compounds in the present invention that may not be properly digested by the user's body that could lead to harmful byproducts to the user.

Although the invention has been explained in relation to its preferred embodiment, it is to be understood that many other possible modifications and variations can be made without departing from the spirit and scope of the invention as hereinafter claimed.

What is claimed is:

1. A medicinal composition for total body detoxification comprises:
a quantity of activated charcoal;
a quantity of dietary supplements; and
the quantity of activated charcoal and the quantity of dietary supplements being heterogeneously mixed into an orally-administrable composition.

2. The medicinal composition for total body detoxification, as claimed in claim 1, wherein the orally-administrable composition is between 280 milligrams (mg) and 320 mg.

3. The medicinal composition for total body detoxification, as claimed in claim 1, wherein the quantity of activated charcoal is between 82 percent by weight (wt %) and 95 wt % of the orally-administrable composition.

4. The medicinal composition for total body detoxification, as claimed in claim 1, wherein the quantity of dietary supplements is cyanocobalamin.

5. The medicinal composition for total body detoxification, as claimed in claim 4, the cyanocobalamin is approximately 0.32 percent by weight (wt %) of the orally-administrable composition

6. The medicinal composition for total body detoxification, as claimed in claim 1, wherein the quantity of dietary supplements is selected from the group consisting of a quantity of sodium chloride, a quantity of potassium chloride, a quantity of magnesium oxide, and combinations thereof.

7. The medicinal composition for total body detoxification, as claimed in claim 6:
wherein the quantity of dietary supplements is a combination of the quantity of sodium chloride, the quantity of potassium chloride, and the quantity of magnesium oxide;
the quantity of sodium chloride being approximately 1.7 wt % of the orally-administrable composition;
the quantity of potassium chloride being approximately 3.5 wt % of the orally-administrable composition; and
the quantity of magnesium chloride being approximately 3.2 wt % of the orally-administrable composition.

8. The medicinal composition for total body detoxification, as claimed in claim 1, comprises:
a quantity of turmeric root powder;
the quantity of turmeric root powder being heterogeneously mixed into the orally-administrable composition

9. The medicinal composition for total body detoxification, as claimed in claim 1, wherein the quantity of turmeric root powder is approximately 16 percent by weight (wt %) of the orally-administrable composition.

10. The medicinal composition for total body detoxification, as claimed in claim 1, wherein the quantity of activated charcoal is derived from coconuts.
